(19) United States
(12) Patent Application Publication
(10) Pub. No.: US 2005/0064076 A1
(43) Pub. Date: Mar. 24, 2005

(54) Method of measuring volatile components of foods

(75) Inventors: Arthur G. Mateos, Schwenskville, PA (US); David Bolotin, Croydon, PA (US); Jose H. Flores, San Pedro Garza Garcia N.L (MX); Salvador Garcia, Lakeland, FL (US)

Correspondence Address: WOOD, HERRON & EVANS, LLP, 2700 CAREW TOWER, 441 VINE STREET, CINCINNATI, OH, 45202, US

(73) Assignee: FMC Technologies, Inc.

(21) Appl. No.: 10/667,729

(22) Filed: Sep. 22, 2003

(51) Int. Cl. G01N 33/02

(52) U.S. Cl. 426/231

(57) ABSTRACT

Methods for determining the level of volatile components, such as essential oils, in a food composition, such as a food processing stream, by analysis of a vapor containing the component. The vapor may be transferred from the headspace of a food processing stream to an analyzing station where the levels of volatile components are determined. Alternatively, the vapor may be generated by sparging a sample of the food or related processing stream for analysis thereof. Analysis of the components maybe performed by spectroscopic methods on a continual or periodic basis.

### FIELD OF THE INVENTION

The invention generally relates to measuring the level of volatile components of foods and, in particular, during the processing of food products.

### BACKGROUND OF THE INVENTION

Volatile components, and particularly essential oils, of foods and their products are useful in many industries. For example, essential oils of fruit and vegetable products are useful as additives to convey fragrance, essence, flavor and other characteristics to foods, beverages, cosmetics, pharmaceuticals and other products. For economic recovery and use of these components, it is desirable to measure their levels in foods and in food products. Additionally, during the processing of certain foods containing volatile components, federal regulations require specific volatile components to be measured, often on a periodic basis and/or at each stage of the process, for commercialization and/or sale of the food product or by-product. Accordingly, there is a need to measure and determine the level of such volatile components in foods and/or during the processing of related food products.

Many methods have been developed for measuring volatile components, and particularly components of essential oils, in foods and/or in related food products. For example, the Scott-Veldhuis distillation-bromate titration procedure has been used for measuring limonene and other less concentrated volatiles, such as diacetyl and furfural, for evaluating the peel oil content of citrus products. However, this method involves the use of bromine under acidic conditions to quantify the levels of the limonene in the product. Such acid and dangerous chemicals compromise user safety and limit application of this method. In addition, this method is time consuming, uses flammable alcohol, and does not distinguish limonene from certain other volatiles, which can interfere with an accurate determination of the limonene in the citrus product.

The Clevenger spice oil method utilizes steam distillation to isolate volatile oil components from a sample of a food product. The isolated components are then measured and quantified to calculate the amount of the component in the food product. While this method eliminates the use of dangerous and hazardous chemicals, such as bromine and acid, it requires the use of steam, which also compromises personal safety and can be dangerous. Moreover, this method is a lengthy process, typically requiring at least one hour to analyze each sample.

Due to their hazards and dangers, the Scott-Veldhuis distillation-bromate titration procedure and the Clevenger spice oil technique are limited in application to measuring levels of volatile components on a small, laboratory scale, and are not especially adaptable or amenable to larger industrial needs. Particularly, these methods would not be amenable to an on-line or an in-line system for measuring volatile components in food processing streams as would be convenient and/or necessary for the production and commercialization of related food products. In addition to being impractical, these methods are relatively complicated and would be costly and generally inefficient for industrial food processing plants, particularly where continuous measurement is required.

Other proposed methods of measuring volatile components in a food product measure the components using known spectroscopic methods.

For example, essential oils of citrus products have been quantified using infra-red spectroscopy by directly removing a sample from the product, preparing the sample for measurement by the infra-red spectroscopy and performing the spectroscopy. However, low solubility of essential citrus oils in aqueous solutions and presence of interfering materials in the sample, such as pulp, make the determination of the level of the essential oils difficult. Particularly, pulp and other more solid materials that interfere with detection of the components by light transmission-absorption techniques, such as infrared spectroscopy, are detrimental to determining the amount of the component in the food product.

Another proposed method of measuring volatile components in food products utilizes attenuated total reflectance (ATR). In this technique, only the liquid in direct contact with the surface of the ATR crystal is measured.

While this technique reduces or eliminates absorption interference due to solid particles, such as pulp, it is not a reliable method for quantifying levels of volatile components because of the nature of the sample matrix. Moreover, this method also presents problems, such as non-uniformity of the sample, inherent with analysis of liquid samples.

Methods that analyze the vapors of the volatile component for determining their levels in a food or food product have also been proposed. For example, one method involves the use of a gas-phase FT-IR method (Fourier Transform Infra-Red Spectroscopy) for rapid analysis of super critical CO₂-extracted volatile caraway fruit oils. However, this method requires a complicated and limited-use extraction procedure in order to analyze the volatile fruit oils. Another method traps vapors of volatile components of the food or food products on porous polymers and then desorbs the vapors from the polymer by heat, provided by a heating jacket around the polymer trap. The desorbed vapors are swept onto a GLC column or condensed in a cold trap for transfer to the GLC column by syringe. Thus, this method is also relatively time consuming and complicated. As such, both methods are not very practical for measuring volatile components, continuously or at regular intervals, in foods and related products in an industrial setting where complexity, cost, and time are important factors to consider.

Yet another proposed method utilizes an electronic sensoring system, called the Znose®, for providing an online quantitative measure of quality for foods, beverages, cosmetics, and other manufactured aromatic products. However, this method is costly and subject to problems and drawbacks associated with electronic systems and failures thereof. Further, this method is not very amenable to continual measurement of volatile components in an in-line industrial food processing stream.

Thus, there remains a need to develop a method of measuring and/or determining the level of a volatile component in a food or related food product in an efficient and effective manner. There is also a need to provide a method of measuring volatile components that is amenable to larger, industrial scale processing plants for manufacture of commercial food products. There is a further need to provide a method for the continual measurement and/or an on-line measurement of the levels of volatile components in a food or related food product. Further, there is a need to provide a method capable of being automated for added efficiency and economy.

### SUMMARY OF THE INVENTION

The present invention provides methods of measuring levels of one or more volatile components in a food composition, such as a natural food, a food product, a food by-product or a food processing stream, while overcoming weaknesses and drawbacks of the previously proposed methods. Volatile components in foods, such as fruits and vegetables, have many uses and their measurement during processing of related products is important. The methods are adaptable for large scales and, in particular, commercial food processing streams in industrial processing plants. In addition, the methods are simple and easy to implement lending to their commercial viability and cost effectiveness. Moreover, the methods are safer than previously proposed methods in that they eliminate the use of toxic and hazardous chemicals and dangerous steam and related heat, and eliminate potential interference and related problems associated with non-volatile components and liquid samples. Accordingly, the methods provide many advantages over the proposed prior art methods, particularly advantages related to the economics of time, cost, and effectiveness of measuring a volatile component in a food or related food product.

In one embodiment of the invention, a method is provided wherein a positively pressurized vapor containing a volatile component is transferred from the headspace of a food processing stream, such as a fruit or vegetable processing stream, into an analyzing station. The vapor is analyzed in the station to determine the level of the volatile component in the processing stream. Analysis is conveniently provided utilizing a single or combination of well-known techniques, such as, infrared spectroscopy (IR), ultraviolet spectroscopy (UV), photoionization, flame ionization, gas chromatography (GC), and gas-liquid chromatography (GLC).

In another embodiment of the invention, a positively pressurized vapor containing a volatile component is generated by sparging a sample of a food composition in a sparging chamber. The positively pressurized vapor is transferred from the sparging chamber to an analyzing station where it is analyzed to determine the level of the volatile component in the composition.

The sample may be transferred from the source of the food composition directly to the sparging chamber. To enhance vapor generation, the sample may be further diluted with a suitable medium, such as pure water, a water-based medium, an organic medium, or an inorganic medium. The sample is then sparged by bubbling an inert gas, such as air, nitrogen, argon, helium, carbon dioxide, and the like, or a combination of gases thereof, through the sample at a suitable flow rate. Flow rates of about 10 ml/min to about 1500 ml/min, are generally suitable to generate the vapor. The pressure in the headspace of the sparging chamber above the sample generally increases as the vapor concentration increases and becomes positive relative to the atmosphere. The positive pressure allows the vapor to be transferred to an analyzing station for analysis of volatile components therein.

In other embodiments, a sample of a food composition, such as a processing stream for a food product or by-product, is transferred to the sparging chamber through an in-line or an on-line connection between the composition and the sparging chamber. In-line connections allow continual determination of the levels of the volatile component in the food composition. Further, an automated in-line connection would allow continual measurement of the volatile component rendering the method more cost efficient, accurate, and effective.

The methods are useful for determining the levels of volatile components of essential oils in processing streams of products and by-products of citrus fruits such as orange, grapefruit, lemon, lime, tangerine, and tangelo, or a combination thereof. Many such components are volatile compounds of specific compound classes, such as aldehydes, alcohols, esters, ketones, and terpenic hydrocarbons. For example, volatile components of essential oils in a citrus product or citrus by-product, such as d-limonene and carvone, and other volatile components of foods and food products, such as octanal, hexanal, citral, linalool, geraniol, citronellal, pinene, myrcene, terpinene, veral, acetaldehyde, valeraldehyde+2-pentanol, furfural, nonanal, decanal, neral, geranial, perillaldehyde, undecanal, dodecanal, ethanol, 1-butanol, 1-penten-3-ol, 3-methyl-1-butanol, trans-2-hexen 1-ol, 1-heptanol, octanol, terpinen-4-ol, alpha-terpineol, nerol+citronellol, carveol, dodecanol, ethyl acetate, methyl butirate, octyl acetate, terpinyl acetate+citronellyl acetate, neryl acetate, geranyl acetate, ethyl anthranylate, acetone, methyl-vinyl-ketone, ethyl-vinyl-ketone, sabinene, alpha-phellandrene, delta₃-carene, beta-cariophyllene, alpha-humulene, valencene, paradisiol, and combinations thereof, can be measured. The methods also measure derivatives and isomers of volatile components. These components can be measured at each stage of a processing stream of the food product or by-product.

By virtue of the foregoing, the present invention provides methods for measuring the levels of volatile components in food compositions and related processing streams that are effective, efficient, and adapted for industrial level food processing streams. Particularly, the methods are adapted for on-line or in-line measurements, optionally performed with assisting automation, thereby eliminating the manpower otherwise required for step-by-step measurement in each stage of the process. These and other benefits and advantages of the present invention shall be further appreciated in light of the following drawings and detailed description of exemplary embodiments of the invention.

### BRIEF DESCRIPTION OF THE DRAWINGS

FIG. 1 is a schematic representation of an exemplary embodiment of the method for determining the level of a volatile component in a composition;

FIG. 2 is a schematic representation of another exemplary embodiment of the method of determining the level of a volatile component in a food processing stream;

FIG. 3 is an FT-IR spectrum of lemon oil containing limonene; and

FIG. 4 is a graph illustrating the percentage of limonene in orange juice measured by the Scott-Veldhuis method, and the percentage of limonene vapor measured by a method of the present invention.

### DETAILED DESCRIPTION OF EXEMPLARY EMBODIMENTS OF THE INVENTION

The present invention provides methods for determining levels of one or more volatile components in a food composition, such as in a food processing stream, without the accompanying weaknesses and drawbacks of previously proposed methods described in the Background of the Invention. Volatile components, and particularly essential oils, are present in natural foods, such as fruits and vegetables, leafy foods such as herbs, and the like, and generally in processed food products and by-products manufactured from natural foods. By-products, such as degradation products of foods, may also contain these essential oils. The term “food composition”, as used herein, is intended to refer to natural foods, processed foods and foods in any stage during the production of processed food products and by-products. Thus, “food composition” encompasses food processing streams. The term “processing stream”, as used herein, is intended to refer to any state or flow of food related to the processing or production of a food product or by-product. Examples of a processing stream include, without limitation, a food product stream, a food by-product stream, and a waste stream.

For determining the level of a volatile component in a food composition, the methods include transferring a positively pressurized vapor containing the volatile component from the headspace of the composition into an analyzing station, and analyzing the positively pressurized vapor to determine the level of the volatile component in the composition. One of the advantages of generating a positively pressurized vapor is that the positive pressure enables transfer of the vapor directly into the analyzing station. This eliminates the need for a vapor carrier, such as the polymer trap proposed in the prior art method, and also eliminates complicated and tedious extraction procedures thereby rendering the present methods more efficient with respect to time and cost and more effective than previously proposed methods.

As mentioned, the methods may be used to measure volatile components in food processing streams. In an enclosed processing stream, the vapors of volatile components present in the headspace above the stream generally have a concentration sufficient to measure and quantify that component in the processing stream. Measurement of the vapor eliminates possible interference of the component analysis by other, non-volatile components such as pulp and the like, which may otherwise be present in a liquid sample. While the vapors may be transferred in any suitable fashion, direct transfer from the source of the food composition containing the volatile component allows measurement in a short period of time and without loss of positive vapor pressure. A direct line thereby reduces manpower otherwise required for the measurement, reduces costs associated with measurement, and allows for continual measurement and determination of the levels of volatile components in a food composition, when necessary. Continual determination of levels of volatile components was generally not feasible or practical in a commercial and/or industrial setting with the previously proposed methods. Whether direct or indirect, the connection between the source of the food composition, such as a food processing stream, and the analyzing station may be an on-line connection or an in-line connection for further economies of scale and ease of implementation.

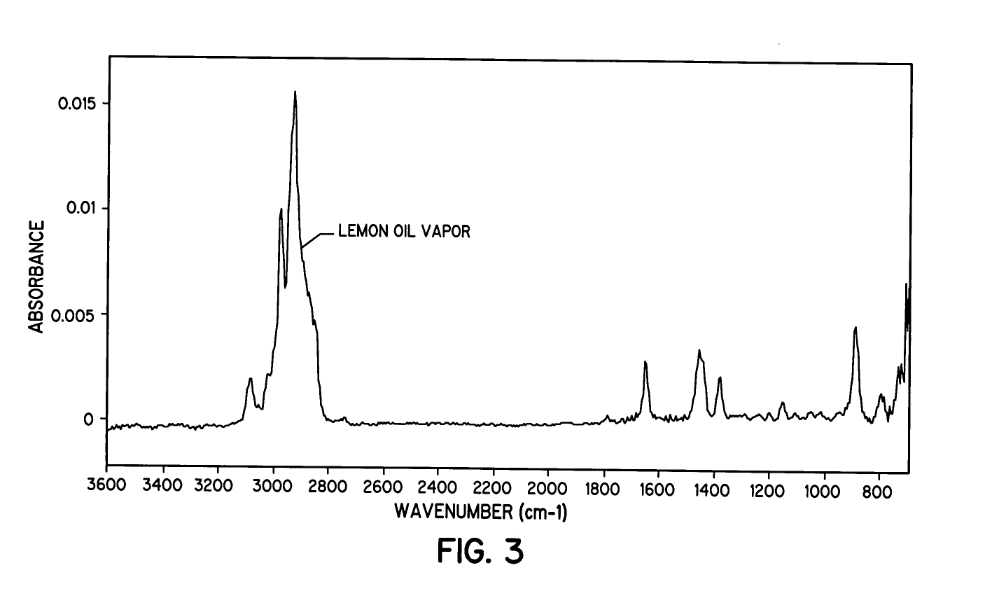

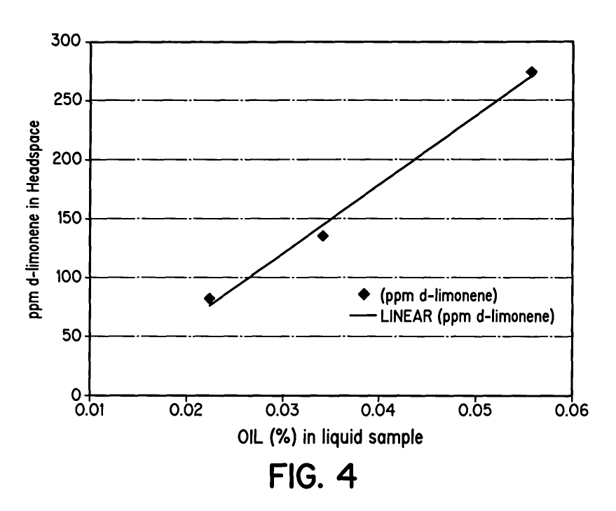

In the analyzing station, the precise amount of the component in the vapor is measured. With this measurement, the amount of the component in the food processing stream or other food composition can be determined in accordance with relevant scientific principles. Known spectroscopic methods and techniques including, without limitation, IR, UV, photoionization, flame ionization, chromatography such as gas-phase chromatography and gas-liquid phase chromatography, and combinations of these techniques may be utilized to detect individual volatile components in the vapor. Analysis of the detected components may be accomplished utilizing known physical and chemical formulae and calculations, as discussed herein with respect to FIGS. 3 and 4.

In one embodiment of the invention, the methods are used to determine the levels of volatile components in processing streams of fruit products and by-products. Processing streams of citrus fruits such as oranges, grapefruit, lemons, limes, tangerine, tangelos, or a combination thereof for example, include volatile components of essential oils in particular, contained in the citrus fruit. Many such components are volatile compounds of specific compound classes, such as aldehydes, alcohols, esters, ketones, and terpenic hydrocarbons. Examples of measurable components of essential oils include, without limitation, d-limonene, carvone, octanal, hexanal, citral, linalool, geraniol, citronellal, pinene, myrcene, terpinene, veral, acetaldehyde, valeraldehyde+2-pentanol, furfural, nonanal, decanal, neral, geranial, perillaldehyde, undecanal, dodecanal, ethanol, 1-butanol, 1-penten-3-ol, 3-methyl-1-butanol, trans-2-hexen 1-ol, 1-heptanol, octanol, terpinen-4-ol, alpha-terpineol, nerol+citronellol, carveol, dodecanol, ethyl acetate, methyl butirate, octyl acetate, terpinyl acetate+citronellyl acetate, neryl acetate, geranyl acetate, ethyl anthranylate, acetone, methyl-vinyl-ketone, ethyl-vinyl-ketone, sabinene, alpha-phellandrene, delta₃-carene, beta-cariophyllene, alpha-humulene, valencene, paradisiol, and their isomers and combinations. The method allows the essential oil to be measured at any or all of the various stages of the processing of the fruit product. For example, a waste stream containing the essential oil and comprised primarily of water may be analyzed via a vapor transferred therefrom to an analyzing station to detect the volatile component, d-limonene, and measure its level in the waste stream.

In another embodiment of the invention, the methods are used to determine the levels of volatile components in the processing streams of vegetable products and by-products. For example, degradation products, such as hexanal and octanal, of vegetables and vegetable oils can be measured by methods of the invention.

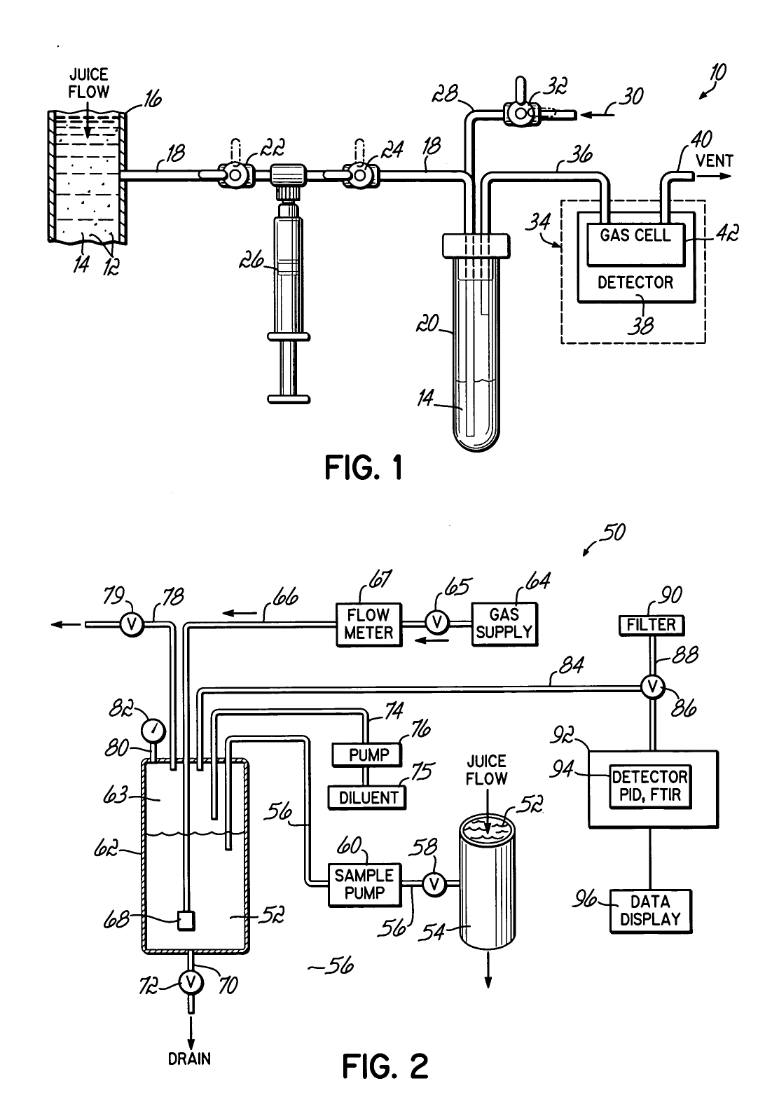

Referring to FIG. 1, there is provided another exemplary embodiment of a method utilizing the principles of sparging to generate a positively pressurized vapor and determine the level of a volatile component 12 in a food composition 14. As shown, system 10 includes a source 16 of food composition 14, containing the volatile component 12, connected through line 18 to a sparging chamber 20. While volatile component 12 is illustrated as insoluble particles in food composition 14, this is for illustration purposes only. Generally, volatile components 12 will be dissolved in food composition 14. Source 16 may be any source, such as the enclosed pipe illustrated in FIG. 1, containing a flowing food processing stream. As shown, line 18 includes two valves 22, 24 and a holding chamber 26, illustrated as a syringe. Valves 22, 24 and holding chamber 26 regulate the flow of a sample of composition 14 from source 16 into the sparging chamber 20. The flow may be provided by a pump or vacuum (not shown) suitably positioned to cause flow of the sample 14 from source 16 to sparging chamber 20.

The term “sparge” is defined as “to introduce air or gas into a liquid” Web Internet Dictionary, 2003. Thus, sparging chamber 20 is fitted with a line 28 through which one or more inert gases 30 are introduced into the sparging chamber 20. Sparging chamber 20 is generally a pressure container into which sample 14 is transferred. Inside sparging chamber 20, sample 14 is sparged with gases 30 to generate a vapor of the volatile components therein in the headspace above sample 14. In other words, gases 30 are bubbled through sample 14 at a gaseous flow rate sufficient to generate the vapor. It is advantageous to bubble in an inert gas, such as nitrogen, argon, helium, carbon dioxide, air, or a combination thereof, through the liquid.

Line 28 includes a valve 32 to regulate the flow and/or amount of gas 30 introduced into sample 14. Sparge line 28 may further include a flow meter (not shown) to measure the flow and pressure of the gas 30 entering sparging chamber 20. Flow rates ranging from about 10 ml/minute to about 1500 ml/minute should be sufficient to safely generate a positively pressurized vapor. In one embodiment, inert gas 30 is bubbled in at a rate of about 750 ml per minute for generating a sufficient concentration of a positively pressurized vapor in the headspace above sample 14. The flow rate will generally depend upon, as appreciated by persons of ordinary skill in the art, the nature and volatility of the components, and upon the concentration of the volatile components in sample 14. Generally, the vapor becomes more concentrated and the pressure in the headspace increases with increased time and rate of sparging sample 14. This vapor will generally contain all the volatile components from sample 14, including those whose levels are to be measured.

Continual sparging of sample 14 creates a positively pressurized vapor in the headspace of sparging chamber 20. The vapor is transferred from sparging chamber 20 to an analyzing station 34 through line 36. Line 36 may be heated to maintain the pressure of the vapors entering analyzing station 34. Line 36 may also include a “dryer” (not shown), such as a membrane or other drying structure to remove some or all of the water vapor content of the vapors being transferred to analyzing station 34. Analyzing station 34 includes a detector 38 therein and a vent 40 to exhaust the vapors and gases 30 from station 34. Detector 38 includes a gas cell 42 which is responsible for detecting the vapors containing the volatile components 12 passing therethrough. Detector 38 relays the detected information to the computer or the “brains” of station 34, or to an outside set of “brains” for computation of the amount of the volatile components 12 both in the vapor and in the food composition 14.

Referring to FIG. 2, there is provided another exemplary embodiment of a method utilizing system 50 for measuring and determining the level of a volatile component in a sample food composition 52. Sample composition 52 is from a citrus product processing stream 54 containing juice and an oil emulsion therein. As shown, sample 52 from the processing stream is transferred through a transfer line 56, having a valve 58 to control the amount of sample 52 transferred to a sparging chamber 62. A pump 60 assists with the transfer of sample 52. One or more sparging gases from gas supply 64 are introduced into sample 52 such as through sparge line 66. Sparge line 66 includes a valve 65 to control the flow of gases from supply 64, and a flow meter 67 to monitor the gas flow. Sparge line 66 terminates in a sparging frit 68 inside sparging chamber 62. Sparging chamber 62 generally includes a drain line 70, having a drain valve 72, to drain sample 52 after the vapor has been transferred and/or analyzed.

As shown, sparging chamber 62 further includes other inlet and outlet ports related to the generation and transfer of a positively pressurized vapor from the headspace 63 of sample 52 in sparging chamber 62 to analyzing station 92. Particularly, port 74 allows a diluent 75 into sparging chamber 62 to further dilute sample 52 to enhance generation of vapors from sample 54. Pump 76 pumps diluent 75 into sparging chamber 62. Exhaust line 78 allows removal of vapors out of sparging chamber 62 into the atmosphere to reduce and regulate the pressure created in the headspace 63 to a desired level or within a desired range. Exhaust line 78 is generally closed via valve 79. Port 80 is fitted with a pressure gauge 82 to further regulate the vapor pressure within sparging chamber 62.

Sample 52 in sparging chamber 62 may be highly concentrated and, therefore, to minimize interference with component analysis and to generate sufficiently pressurized and concentrated vapors, sample 52 may be diluted with a suitable diluent 75. Diluents such as water, water-based media, organic media, inorganic media, and combinations thereof may be added to sample 52. A water-based media may include for example, water-miscible organic or inorganic compounds therein. Mixtures containing water and an alcohol, such as methanol or ethanol, are suitable. Diluent 75 can also be tailored towards the properties of the volatile components. For example, hydrophobic solvents such as hexane, pentane, octane, and other non-polar organic solvents may be added to dissolve essential oils and other volatile lipophilic components in sample 52. Diluent 75 should be chosen in accordance with the method of component analysis and method of detection used. For example, photoionization detectors are immune to the influence and interference from methanol and, therefore, methanol is a suitable sample diluent for analysis with a photoionization detector.

As shown, there is a transfer line 84 between sparging chamber 62 and analyzing station 92 through which the positively pressurized vapor from headspace 63 is transferred to analyzing station 92. Transfer line 84 includes a valve 86 connected to vent line 88 having a charcoal filter 90. Charcoal filter 90 prevents outside contaminants from entering system 50, and particularly into line 84, to contaminate the vapor entering analyzing station 92 from headspace 63 of sparging chamber 62. Transfer line 84 continues to analyzing station 92.

Vent line 88 may also serve to decrease the concentration of vapors being transferred from sparging chamber 62 to analyzing station 92, so as to prevent vapors, otherwise too concentrated and/or pressurized for a detector to detect without going off-scale, from entering detector 94. Alternatively, the vapor concentration may be decreased by “splitting” whereby only a portion of the vapor generated in sparging chamber 62 is removed from sparging chamber 62 and transferred to analyzing station 92. It should be appreciated that depending upon the rate of vapor transfer from sparging chamber 62 to analyzing station 92 and the rate of gas introduction from supply 64 into sparging chamber 62, there may be a vacuum created in headspace 63 of sparging chamber 62.

The analyzing station 92 may include any number and type of analyzing instruments as necessary for measuring the levels of volatile components in the vapor of sample 52. Analysis of the vapor may be accomplished utilizing conventional techniques known in the art. For example, suitable techniques include infrared spectroscopy, ultraviolet spectroscopy, photoionization, flame ionization, and chromatography. Chromatography may either be gas-liquid chromatography or a gas-phase chromatography system. Each technique will generally include use of a detector 94 wherein the individual components of the volatile vapor are detected and charted or plotted in a corresponding spectrum. Detector 94 will generally have a exhaust (not shown) for the flow of gas and vapor out of detector 94.

The spectral information generated by detector 94 may be analyzed by appropriate programs utilizing the data in relevant physical and chemical formulae to calculate the levels of the individual components of the vapor. For example, d-limonene in the vapor of a product or by-product stream of a citrus fruit may be measured using an FT-IR or photoionization detector wherein the detected information is sent as a data output 4-20 milliamp signal, which may also be displayed on the detector screen or other data display 96, such as an independent computer terminal inside or outside of analyzing station 92.

Referring to FIG. 3, there is shown an FT-IR spectrum of vapor, containing d-limonene, transferred from the headspace of lemon oil, sparged in a sparging chamber, and measured at room temperature by an FT-IR detector. As shown, the limonene spectrum is relatively clear of any interference from the sparged, lemon oil sample matrix and sufficient for quantitative analysis of the d-limonene in the oil.

The spectral data of the volatile component may be analyzed by methods known to those skilled in the art to quantify the component in the food. For example, under equilibrium conditions, the relation between a vapor and its concentration in the liquid, from which the vapor was obtained, is expressed by Raoult's Law: P=FP₀. P is the equilibrium vapor pressure, P₀ is the inherent vapor pressure of the pure volatile component, and F is the fractional saturation of the sample composition (usually a solution). F is defined as F=C/C₀ where C=concentration of the volatile compound, and C₀ is the concentration at saturation. The equilibrium vapor (P) and concentration (C) of the volatile component in the sample are related by equation: P=(P₀/C₀)C which is commonly expressed as Henry's Law, P=KC where K=(P₀/C₀). Thus, K is the partition coefficient of the volatile component between its liquid state in the sample composition and its vapor state in the headspace above the sample.

The partition coefficient provides the correlation between the concentration and amount of the component in the vapor state (headspace) and its amount in the sample composition, whose concentration can be determined using calculations and methods known to those skilled in this art. More particularly, there is direct correlation between the concentration of a volatile component in a liquid sample and the concentration of that component in the vapor in the headspace above the sample. For example, and with reference to FIG. 4, there is illustrated the relationship between percent concentration of limonene oil in sample of orange juice, as measured by the Scott-Veldhuis method, and the percent concentration of limonene vapor in the headspace above the orange juice sample as measured by one method of the invention utilizing a flame ionization detector. As shown, the relationship between the concentration in the sample liquid and that in the headspace (vapor) is linear and directly proportional. Thus, measurement of levels of volatile components by vapor concentrations in headspace provides a direct correlation to the concentration in the sample. Accordingly, in conjunction with known methods or the method of analysis described above, concentrations of volatile components in the sample of the food composition can be determined, and levels in the food composition as a whole can be quantified.

Another method of deriving the final level of a volatile component in the sample is by comparison of spectral data of the vapor. For example, the spectral data from sample 52 may be mapped against the pure spectrum of the individual component of known concentration to identify and analyze the level of that component in sample 52. More particularly, the area under the curve in the spectrum may be used to calculate the concentration of the volatile component in the vapor by known methods and to ascertain the concentration and amount of the component in sample 52.

Typically, however, each essential oil of a sample composition contains multiple volatile components with each component having different inherent vapor pressures. Therefore, the vapor composition in the headspace of the constituents will not exactly resemble that of the liquid phase of the sample composition. In such a case of a multi-component composition having multiple volatile constituents with different vapor pressures, a depletion rate analysis may be performed to analyze the amounts of the individual components.

In this analysis, a static volume of the liquid sample is sparged while monitoring the depletion of the oil constituents through analysis of the removed vapor. By initially determining the depletion rate of oil from a liquid sample, under a specific set of conditions, the exponential decay function for the evaporation of oil can be established. This decay function may generally be expressed by the following formula:
Cᵥ(t)=Cᵥ(0) 2^{-at}
where Cᵥ and Cᵥ(0) are vapor phase concentrations, a is the depletion rate constant and t is time.

The measurement of two or more points on the depletion curve allows one to determine both the initial vapor phase concentration Cᵥ(0) and the value of a. The partitioning function K is related to the rate constant a by the expression,
K=a(V_{w}/f)(RT/Wₘ)
where: V_{w} is the air flow rate, R is equal to 0.08206 (a known constant), T is the temperature of the vapor (° K), and Wₘ is the molecular weight of the oil. Knowing the partition function (K), one can determine the initial concentration of oil in the sample liquid or water, where water is the diluent, from the measured initial vapor concentration using the following formula:
C(0)=(Cᵥ(0)/a)(fWₘ/V_{w})
W_{m,} in a mult-volatile component oil will be the molecular weight representing the average molecular weights of the different constituents of oil. Persons of ordinary skill in the art can readily use these formulae to determine the levels of the volatile components in the sample composition and extrapolate that amount to determine the concentration and/or amount in the source of the composition. The comparison method is also useful for determining amounts of multiple volatile components in a sample.

FIGS. 1 and 2 illustrate an on-line system that may be adapted for measurement of levels of volatile components in an industrial scale food processing stream. The term “on-line”, as used herein, generally refers to a direct feed line or a connection between the source of the food composition, such as a processing stream, and the sparging chamber or the analyzing station (in those embodiments without the sparging chamber). In either on-line system, it is advantageous to have a means to introduce (open) or shut down or restrict (close) the flow of the sample composition, or positively pressurized vapor, as desired by the user. Particularly, as shown, the sample transfer lines of FIGS. 1 and 2 include a valve to regulate and/or control the flow of sample food composition into the sparging chamber. Such an on-line connection would provide a system, adaptable for automation, for periodic measurement of the levels of the volatile components in the processing stream.

Similarly, valves 22, 24 and/or holding chamber 26 of FIG. 1, for example, may be omitted to provide an in-line connection where sample compositions from the food processing stream are continuously provided to the sparging chamber for analysis thereof. The term “in-line”, as used herein, refers generally to a continuous feed line providing sample compositions into a sparging chamber, or positively pressurized vapors into an analyzing station (in embodiment without a sparging chamber), from the source of the food composition such as the processing stream illustrated in FIGS. 1 and 2. Such an in-line connection would allow for continuous determination of the levels of the volatile components where required.

A sample composition of the processing food stream may be introduced to either of an on-line or an in-line system by way of an injection port through which the sample is removed via syringe or other similar methods into a feedline pipe. The feedline pipe may include ports in which the sample may be diluted with a suitable diluent while being transferred into a sparging chamber. Samples analyzed by such on-line and in-line systems which may be monitored through a computer system, for constant determination of the levels of volatile components. Thus, the present invention provides methods adaptable for larger, commercial use in addition to smaller, laboratory scale measurements.

Between sample analyses, the system should be cleaned and purged to remove all sample residue which may otherwise contaminate the new sample and/or provide false measurements for the volatile components.

Flushing the system with air or inert gas is particularly important to purge the detector, especially the detecting cell, from contaminants. To this end, pure air from the atmosphere may be introduced into the detector, advantageously through an air-filtering device such as the charcoal filter 90 shown in FIG. 2, and out the detector exhaust. Similarly, the sparging chamber may be purged of contaminants by simply rinsing the chamber with water or suitable solvents to clean the chamber, and draining the water from the chamber.

Thus, there is provided methods for measuring the levels of volatile components in food compositions, and in particular, in food processing streams. Measuring levels of volatile components of essential oils, such as d-limonene, carvone, and others, in fruit and vegetable processing streams is important for compliance with federal standards and commercialization of final products and by-products from these processing streams. Further, many essential oils are utilized as additives in various industries, and therefore, their levels in foods are frequently determined to decide whether or not it is at the desirable level and/or meets the governmental food regulations. The methods are adaptable for on-line or in-line measuring systems, and are commercially feasible while measuring the levels of volatile components in foods and related products in an efficient, effective, convenient and safe manner.

While the present invention has been illustrated by a description of exemplary embodiments and while these embodiments have been described in some detail, it is not the intention of the Applicants to restrict or in any way limit the scope of the appended claims to such detail. Additional advantages and modifications will readily appear to those skilled in the art. For example, while the analysis herein describes four different methods of detecting the component in the vapor of the food composition, the method is not so limited and may include other analytical techniques, known in the art. The various features of the invention may be used alone or in numerous combinations depending on the needs and preferences of the user. This has been a description of the present invention, along with the exemplary methods of practicing the present invention as currently known. However, the invention itself should only be defined by the appended claims.

1. A method of determining the level of a volatile component in a food processing stream, the method comprising:
transferring a positively pressurized vapor containing the volatile component from the headspace of the processing stream into an analyzing station; and
analyzing the positively pressurized vapor to determine the level of the volatile component in the processing stream.

2. The method of claim 1 wherein the processing stream is selected from the group consisting of a fruit product processing stream, a fruit by-product processing stream, a vegetable product processing stream and a vegetable by-product processing stream.

3. The method of claim 1 wherein the volatile component is an essential oil of at least one of a citrus product and a citrus by-product.

4. The method of claim 1 wherein the volatile component is a compound of a class selected from one of an aldehyde, an ester, a ketone and a terpenic hydrocarbon.

5. The method of claim 1 wherein the volatile component is selected from the group consisting of limonene, carvone, octanal, hexanal, citral, linalool, geraniol, citronellal, pinene, myrcene, terpinene, veral, acetaldehyde, valeraldehyde, 2-pentanol, furfural, nonanal, decanal, neral, geranial, perillaldehyde, undecanal, dodecanal, ethanol, 1-butanol, 1-penten-3-ol, 3-methyl-1-butanol, trans-2-hexen 1-ol, 1-heptanol, octanol, terpinen-4-ol, alpha-terpineol, nerol+citronellol, carveol, dodecanol, ethyl acetate, methyl butirate, octyl acetate, terpinyl acetate, citronellyl acetate, neryl acetate, geranyl acetate, ethyl anthranylate, acetone, methyl-vinyl-ketone, ethyl-vinyl-ketone, sabinene, alpha-phellandrene, delta₃-carene, beta-cariophyllene, alpha-humulene, valencene, paradisiol, isomers thereof, and combinations thereof.

6. The method of claim 1 wherein analyzing the vapor comprises a technique selected from the group consisting of an infrared spectroscopy, ultraviolet spectroscopy, photoionization, flame ionization, gas chromatography, and a combination thereof.

7. A method of determining the level of a volatile component in a composition, including at least one of a fruit and a vegetable, the method comprising:
sparging a sample of the composition in a sparging chamber and thereby generating a positively pressurized vapor containing the volatile component;
transferring the positively pressurized vapor into an analyzing station; and
analyzing the vapor to determine the level of the volatile component in the composition.

8. The method of claim 7 further comprising:
transferring the sample from a processing stream of the composition to the sparging chamber through an online connection between the processing stream and the sparging chamber.

9. The method of claim 7 further comprising:
transferring the sample from a processing stream of the composition to the sparging chamber through an inline connection between the processing stream and the sparging chamber.

10. The method of claim 7 further comprising transferring the sample from a waste stream of the composition to the sparging chamber.

11. The method of claim 7 wherein analyzing the vapor comprises a technique selected from the group consisting of an infrared spectroscopy, ultraviolet spectroscopy, photoionization, flame ionization, gas chromatography, a combination thereof.

12. The method of claim 7 wherein sparging the sample comprises bubbling an inert gas including at least one air, nitrogen, argon, helium, carbon dioxide, through the sample.

13. The method of claim 12 wherein the gas is bubbled at a flow rate in the range from about 10 ml/min to about 1500 ml/min.

14. The method of claim 7 wherein the volatile component is an essential oil of at least one of a citrus product and a citrus by-product of a fruit.

15. The method of claim 9 wherein the volatile component is a degradation product of a vegetable oil.

16. The method of claim 7 wherein the volatile component is a compound of a class chosen from one of an aldehyde, an alcohol, an ester, a ketone, and a terpenic hydrocarbon.

17. The method of claim 7 wherein the volatile component is selected from the group consisting of limonene, carvone, octanal, hexanal, citral, linalool, geraniol, citronellal, pinene, myrcene, terpinene, acetaldehyde, valeraldehyde, 2-pentanol, furfural, nonanal, decanal, neral, geranial, perillaldehyde, undecanal, dodecanal, ethanol, 1-butanol, 1-penten-3-ol, 3-methyl-1-butanol, trans-2-hexen 1-ol, 1-heptanol, octanol, terpinen-4-ol, alpha-terpineol, nerol, citronellol, carveol, dodecanol, ethyl acetate, methyl butirate, octyl acetate, octyl acetate, terpinyl acetate, citronellyl acetate, neryl acetate, geranyl acetate, ethyl anthranylate, acetone, methyl-vinyl-ketone, ethyl-vinyl-ketone, sabinene, alpha-phellandrene, delta3-carene, beta-cariophyllene, alpha-humulene, valencene, paradisiol, isomers thereof, and combinations thereof.

18. The method of claim 7 further comprising diluting the sample with a liquid including at least one of water, an aqueous-based solvent, an organic solvent, an inorganic solvent, and a combination thereof, prior to sparging the sample.

19. A method of determining the level of a volatile oil in a citrus processing stream, the method comprising:
transferring a sample containing the volatile oil from the processing stream into a sparging chamber;
sparging the sample and thereby generating a positively pressurized vapor containing the volatile oil;
transferring the positively pressurized vapor into an analyzing station; and
analyzing the vapor to determine the level of the volatile oil in the processing stream.

20. The method of claim 19 wherein transferring the sample comprises transferring the sample from the processing stream to the sparging chamber through an online connection between the processing stream and the sparging chamber.

21. The method of claim 19 wherein transferring the sample comprises transferring the sample from the processing stream to the sparging chamber through an inline connection between the processing stream and the sparging chamber.

22. The method of claim 19 wherein transferring a sample from the processing stream comprises transferring a sample from a waste stream.

23. The method of claim 19 further comprising diluting the sample with a liquid including at least one of water, an aqueous-based solvent, an organic solvent, an inorganic solvent, prior to sparging the sample.

24. The method of claim 19 wherein sparging the sample comprises bubbling an inert gas including at least one of air, nitrogen, argon, helium, carbon dioxide, through the sample.

25. The method of claim 24 wherein the gas is bubbled at a flow rate in the range from about 10 ml/min to about 1500 ml/min.

26. The method of claim 19 wherein analyzing the vapor comprises a technique selected from the group consisting of an infrared spectroscopy, ultraviolet spectroscopy, photoionization, flame ionization, gas chromatography, and a combination thereof.

27. The method of claim 19 wherein the volatile oil is an essential oil of at least one of a citrus product and a citrus by-product of a citrus fruit.

28. The method of claim 19 wherein the volatile oil includes a compound of at least one of a citrus product and a citrus by-product.

29. The method of claim 19 wherein the volatile oil includes a component selected from the group consisting of limonene, carvone, octanal, hexanal, citral, linalool, geraniol, citronellal, pinene, myrcene, terpinene, veral, acetaldehyde, valeraldehyde, 2-pentanol, furfural, nonanal, decanal, neral, geranial, perillaldehyde, undecanal, dodecanal, ethanol, 1-butanol, 1-penten-3-ol, 3-methyl-1-butanol, trans-2-hexen 1-ol, 1-heptanol, octanol, terpinen-4-ol, alpha-terpineol, nerol, citronellol, carveol, dodecanol, ethyl acetate, methyl butirate, octyl acetate, terpinyl acetate, citronellyl acetate, neryl acetate, geranyl acetate, ethyl anthranylate, acetone, methyl-vinyl-ketone, ethyl-vinyl-ketone, sabinene, alpha-phellandrene, delta₃-carene, beta-cariophyllene, alpha-humulene, valencene, paradisiol, isomers thereof, and combinations thereof.

30. The method of claim 19 wherein the citrus processing stream includes at least one of an orange, grapefruit, lemon, lime, tangerine, tangelo, product and by-product.
